(19) United States
(12) Patent Application Publication
(10) Pub. No.: US 2026/0294855 A1
(43) Pub. Date: Oct. 1, 2026

(54) PHARMACEUTICAL COMPOSITION COMPRISING IBUPROFEN

(71) Applicant: Ioulia TSETI, Kifissia Attikis (GR)

(72) Inventor: Ioulia TSETI, Kifissia Attikis (GR)

(21) Appl. No.: 19/489,442

(22) PCT Filed: May 31, 2024
(86) PCT No.: PCT/GR2024/000014

§ 371 Date: Dec. 2, 2025

(30) Foreign Application Priority Data
Jun. 2, 2023 (GR) 20230100444
Jul. 27, 2023 (GR) 20230100623
Sep. 13, 2023 (GR) 20230100733

(51) Int. Cl. A61K 31/192; A61K 9/00; A61K 9/08; A61K 9/16; A61K 9/20; A61K 9/46; A61K 31/167; A61K 31/375; A61K 47/02; A61K 47/10; A61K 47/12; A61K 47/18; A61K 47/20; A61K 47/22; A61K 47/26; A61K 47/36; A61K 47/38; A61K 47/40

(52) U.S. Cl. CPC A61K 31/192; A61K 9/0007; A61K 9/0019; A61K 9/0095; A61K 9/08; A61K 9/1611; A61K 9/1617; A61K 9/1635; A61K 9/1652; A61K 9/2009; A61K 9/2013; A61K 9/2027; A61K 9/2059; A61K 31/167; A61K 31/375; A61K 47/02; A61K 47/10; A61K 47/12; A61K 47/183; A61K 47/20; A61K 47/22; A61K 47/26; A61K 47/36; A61K 47/38; A61K 47/40

(57) ABSTRACT

The present invention refers to pharmaceutical compositions comprising ibuprofen or a pharmaceutically acceptable derivative thereof and one or more solubilizing agents in the form of A) aqueous solution for use in intravenous administration comprising ibuprofen of either the sodium or lysinate salt, paracetamol and preferably one or more cyclodextrins or B) aqueous solution for use in intravenous administration wherein the one or more solubilizing agents are one or more essential amino acids and optionally one or more cyclodextrins or C) composition for use in oral administration, which comprises paracetamol and cyclodextrin, wherein said composition is in the form of either i) effervescent tablets or effervescent granules or ii) oral suspension or syrup or oral solution or iii) soft capsules.

### DESCRIPTION OF THE INVENTION

The present invention refers to pharmaceutical compositions comprising ibuprofen or a pharmaceutically acceptable derivative thereof and one or more solubilizing agents in the form of
A) aqueous solution for use in intravenous administration comprising ibuprofen of either the sodium or lysinate salt, paracetamol and preferably one or more cyclodextrins or
B) aqueous solution for use in intravenous administration wherein the one or more solubilizing agents are one or more essential amino acids and optionally one or more cyclodextrins or
C) composition for use in oral administration, which comprises paracetamol and cyclodextrin, wherein said composition is in the form of either i) effervescent tablets or effervescent granules or ii) oral suspension or syrup or oral solution or iii) soft capsules.

Ibuprofen is a non-steroidal anti-inflammatory drug with anti-inflammatory, analgesic and antipyretic properties, which is indicated for the treatment of fever and pain, especially when associated with inflammation [Irvine J., Afrose A., Islam N. Formulation and delivery strategies of ibuprofen: challenges and opportunities. Drug Dev Ind Pharm. 2018; 44 (2): 173-183]. The combination of ibuprofen with paracetamol is used to enhance the analgesic effect, as for example in musculoskeletal pain [Bettiol A., Marconi E., Vannacci A., Simonetti M., Magni A., Cricelli C., Lapi F. Effectiveness of ibuprofen plus paracetamol combination on persistence of acute musculoskeletal disorders in primary care patients. Int J Clin Pharm. 2021; 43 (4): 1045-1054].

The low solubility of ibuprofen provokes dissolution problems in the cases of the injectable forms, either with paracetamol or without paracetamol.

Document CN101991540A refers to an ibuprofen injection, comprising ibuprofen as a main drug and pharmaceutically acceptable auxiliary materials, wherein said pharmaceutically acceptable auxiliary materials include cyclodextrins or cyclodextrin derivatives and water-soluble excipients. The content is 0.5% to 20% by weight of ibuprofen, 10% to 60% of cyclodextrins or cyclodextrin derivatives, 1% to 40% of water-soluble excipients, and the rest is water. However, it does not mention the use of specific solubilizing agents, such as basic amino acids, for example histidine or arginine.

Document TW201617066A relates to a method for preparing a combination product of ibuprofen and paracetamol, comprising the step of dissolving 2.8 to 3.2 mg ibuprofen and 9.8 to 10.2 mg paracetamol expressed as per ml of said composition in an aqueous solvent of pH 6.3-7.3, without however referring to the dissolution of even higher concentrations of ibuprofen, for example of 100 mg/ml of the present invention, due to the presence of solubilizing agents.

Document EP2635269A1 refers to an intravenous composition, which comprises ibuprofen and paracetamol in a dose of: a) about 125 mg to about 175 mg ibuprofen in combination with about 475 mg to about 525 mg paracetamol or b) about 275 mg to about 325 mg ibuprofen in combination with about 975 mg to about 1025 mg paracetamol. The above amounts are dissolved in 100 ml of water. However, it does not mention the dissolution of even higher concentrations of ibuprofen, for example of 100 mg/mL of the present invention, due to the presence of solubilizing agents.

Documents EP2389923B1 & EP2277546B1 refer to injectable paracetamol compositions comprising cyclodextrins as stabilizing agents. However, it does not mention the simultaneous dissolution of ibuprofen other than paracetamol.

Document EP3169307B1 relates to compositions of paracetamol and ibuprofen suitable for injectable administration, wherein the pH of the composition is from 6.3 to 7.3 and said compositions comprise from 2.8 to 3.2 mg of ibuprofen and from 9.8 to 10.2 mg of paracetamol expressed per ml of said composition. However, it does not refer solubilizing agents that could contribute to the dissolution of even higher concentrations of ibuprofen, for example of 100 mg/ml of the present invention, due to the presence of solubilizing agents.

Document EP3612157A1 relates to compositions comprising ibuprofen and paracetamol, suitable for injectable administration, which additionally comprise dissolved oxygen at a maximum concentration of 1.0 ppm in a closed container, pH from 6.3 to 7.3, one or more antioxidants, one or more isotonic agents and one or more buffering agents, wherein the concentration of paracetamol is 10 mg/ml and the concentration of ibuprofen is 2 to 4 mg/mL. However, it does not refer to solubilizing agents that could contribute to the dissolution of even higher concentrations of ibuprofen, for example of 100 mg/ml of the present invention, due to the presence of solubilizing agents.

Moreover, the low solubility of ibuprofen, which affects its dissolution and absorption, and therefore its bioavailability, is a major problem during the development of effective formulations for the oral administration of the ibuprofen-paracetamol combination.

Document EP2089013A1 refers to analgesic, antipyretic compositions, for oral dosage, comprising ibuprofen and paracetamol in ratios from 5:19 to 81:95.

Document CN102389423A relates to formulations of ibuprofen sodium in combinations with other active substances, including ibuprofen-paracetamol combination, which are formulated into oral forms such as tablets, dispersible tablets, extended-release tablets, capsules, granules, dry suspensions, suspensions and others similar. The amount of ibuprofen ranges from 12.5 to 1000 mg, preferably from 50 to 500 mg and the amount of paracetamol ranges from 25 to 1000 mg, preferably from 100 to 500 mg.

Document WO2009083759A1 refers to an oral pharmaceutical suspension composition comprising 40-80 mg/5 ml ibuprofen, 100-500 mg/5 ml paracetamol and one or more pharmaceutically acceptable excipients.

Document EP0109281A1 relates to compositions of flubiprofen or ibuprofen or derivatives thereof, paracetamol and a pharmaceutically acceptable carrier, which are formulated into powder or liquid.

Document CN103751158A refers to a pharmaceutical oral suspension composition comprising 40-80 mg/5 ml ibuprofen, 100-500 mg/5 ml paracetamol and one or more pharmaceutically acceptable excipients.

Document EP1129709A2 refers to an effervescent composition comprising ibuprofen and cyclodextrin, but no paracetamol.

Document CN100404025C refers to an effervescent composition comprising paracetamol and cyclodextrin, but no ibuprofen.

However, none of the above documents about formulations for oral administration mentions the triple combination of ibuprofen, paracetamol and cyclodextrin in the composition.

Despite of the achievements already mentioned in the state of the art, due to the increased requirements of quality and safety, there is a continuous need to further improve the stability of the aqueous solution of ibuprofen in the form of either sodium salt or lysinate and paracetamol for use in IV administration, even in high temperatures, for a long time, without the need to keep it in a completely airtight bottle.

However, the combination of ibuprofen in the form of sodium salt or lysinate and paracetamol and preferably cyclodextrin as a solubilizing agent for the preparation of compositions of injectable solutions suitable for intravenous administration has not yet been reported in the state of the art.

Notably, if the skilled person, starting with the product comprising the two active ingredients paracetamol and ibuprofen, wanted to improve the solubility, he would start with the more difficult to be dissolved, which is ibuprofen. Although the state of the art mentions the use of solubilizing agents e.g. cyclodextrins, for the least insoluble, i.e. paracetamol, there is no technical information on how the solubility of a pharmaceutical composition comprising ibuprofen and paracetamol, could be improved.

Despite of the achievements already mentioned in the state of the art, due to the increased requirements of quality and safety, there is a continuous need to further improve the stability of the aqueous solution of ibuprofen for use in IV infusion, even at high temperatures, for a long time, without the need to keep it in a completely airtight bottle.

However, the combination of ibuprofen with histidine and optionally with cyclodextrin for the preparation of compositions of injectable ibuprofen solutions, suitable for intravenous administration, has not been reported in the prior art yet.

Despite of the achievements already mentioned in the state of the art, there is a continuous need for further improvement of pharmaceutical products of ibuprofen in combination with paracetamol for use in oral administration, with an emphasis on pharmaceutical forms other than classic tablets, in order to improve the compliance of patient, by facilitating swallowing.

However, the combination of ibuprofen-paracetamol in orally administered forms including effervescent forms, by using solubilizing agents and specifically cyclodextrin in an appropriate amount, in order to achieve sufficient dissolution and/or suspension of the active substances, has not been referred in the prior art yet.

More specifically, the present invention is defined by the following:
Definition 1. Pharmaceutical composition comprising ibuprofen or a pharmaceutically acceptable derivative thereof and one or more solubilizing agents in the form of
A) aqueous solution for use in intravenous administration comprising ibuprofen of either the sodium or lysinate salt, paracetamol and preferably one or more cyclodextrins or
B) aqueous solution for use in intravenous administration wherein the one or more solubilizing agents are one or more essential amino acids and optionally one or more cyclodextrins or
C) composition for use in oral administration, which comprises paracetamol and cyclodextrin, wherein said composition is in the form of either i) effervescent tablets or effervescent granules or ii) oral suspension or syrup or oral solution or iii) soft capsules.
Definition 2. Pharmaceutical composition according to definition 1, wherein the ibuprofen is preferably in the form of the sodium salt.
Definition 3. Pharmaceutical composition according to any of the definitions 1 to 2, wherein the amount of ibuprofen ranges from 1 to 1000 mg, preferably from 150 to 600 mg or 200 to 500 mg or 300 to 400 mg.
Definition 4. Pharmaceutical composition according to any of the definitions 1 to 3, wherein in the form of aqueous solution A or B, the concentration of equivalent free ibuprofen ranges from 1 to 400 mg/mL, preferably from 2 to 100 mg/mL, more preferably from 3 to 50 mg/mL and even more preferably 3 mg/mL.
Definition 5. Pharmaceutical composition according to any one of definitions 1 to 4, which comprises paracetamol preferably in a ratio of ibuprofen:paracetamol 600 mg:1000 mg or 500 mg:1000 mg or 500 mg:500 mg or 400 mg:1000 mg or 400 mg:500 mg or 300 mg:1000 mg or 300 mg:500 mg or 200 mg:1000 mg or 200 mg:500 mg or 200 mg:200 mg or 200 mg:100 mg or 150 mg:1000 mg or 150 mg:500 mg or 150 mg:200 mg or 150 mg:100 mg.
Definition 6. Pharmaceutical composition according to any of the definitions 1 to 5, wherein when in the form of aqueous solution A, the concentration of paracetamol ranges from 0.20% to 10% m/v, preferably from 0.5% to 1.5% m/v.
Definition 7. Pharmaceutical composition according to any of the definitions 1 to 6, wherein when in the form of aqueous solution A or B, said composition comprises one or more cyclodextrins, preferably hydroxyalkyl-β-cyclodextrins, more preferably 2-hydroxypropyl-β-cyclodextrin, wherein the concentration of the cyclodextrin ranges from 0.2% m/v to 19% m/v, preferably from 0.2% m/v to 6% m/v, more preferably from 0.5% m/v to 3% m/v.
Definition 8. Pharmaceutical composition according to any of the definitions 1 to 6, wherein when in the form of aqueous solution A or B, said composition comprises one or more cyclodextrins, preferably hydroxyalkyl-β-cyclodextrins, more preferably 2-hydroxypropyl-β-cyclodextrin, wherein the concentration of the cyclodextrin ranges from 10 to 2000 mg/mL, preferably from 200 to 1500 mg/mL, more preferably from 350 to 750 mg/mL.
Definition 9. Pharmaceutical composition according to any of the definitions 1 to 8, which comprises one or more essential amino acids, preferably histidine or arginine, more preferably histidine, wherein when said composition is in the form of aqueous solution A or B the concentration of the essential amino acid ranges from 1 to 400 mg/L, preferably from 20 to 300 mg/L, more preferably from 50 to 200 mg/L and even more preferably from 70 to 100 mg/L.
Definition 10. Pharmaceutical composition according to definition 9, wherein when in the form of aqueous solution A or B, said composition comprises one or more essential amino acids, wherein the concentration of said essential amino acid ranges from 0.5 to 200 mg/L, preferably from 10 to 150 mg/L, more preferably from 25 to 100 mg/L and even more preferably from 35 to 50 mg/L and one or more cyclodextrins, wherein the concentration of the cyclodextrin is from 10 to 2000 mg/mL, preferably from 200 to 1500 mg/mL, more preferably from 350 to 750 mg/mL.
Definition 11. Pharmaceutical composition according to any of the definitions 1 to 10, wherein when in the form of aqueous solution A or B, said composition comprises additionally a derivative bearing at least one thiol functional group, which is selected from thioglycerols, cysteine, acetylcysteine, thioglycolic acid and/or salts thereof, dithiothreitol, reduced glutathione, thiolactic acid and/or salts thereof and mercaptoethanesulfonic acid, preferably thioglycerol and particularly preferably monothioglycerol, wherein the concentration of the derivative bearing at least one thiol functional group ranges from 0.001% to 0.5% m/v either from 1.5% to 3% m/v or from 2% to 2.5% m/v.
Definition 12. Pharmaceutical composition according to any of the definitions 1 to 11, wherein when in the form of aqueous solution A or B, said composition comprises a chelating agent selected from EDTA, nitrilotriacetic acid, ethylenediamine-N,N′-dipropionic acid, ethylenediamine-tetra-(methylene phosphoric acid)), 2,2′ (ethylenediamino)-dibutyl acid, bis(2-aminoethyl ether)-N,N,N′ethylene glycol, N′-tetraacetic acid and/or their salts, preferably EDTA, wherein the concentration of the chelating agent ranges from 0.001% to 0.5% m/v either from 1.5% to 3% m/v or from 2% to 2.5% m/v.
Definition 13. Pharmaceutical composition according to any of the definitions 1 to 12, wherein when in the form of aqueous solution A or B, said composition comprises one or more derivatives selected from thiamine salts, preferably thiamine hydrochloride, wherein the concentration of the thiamine salt ranges from 0.001% m/v to 0.2% m/v.
Definition 14. Pharmaceutical composition according to any of the definitions 1 to 13, wherein when in the form of aqueous solution A or B, pH is between 4 and 8, preferably from 6 to 8, more preferably from 7 to 8, such as pH=7.6, said pH is adjusted by using a suitable alkalizing agent, preferably sodium carbonate and/or by at least one acid and its ionized form, said acid is selected from citric, malic, acetate, sorbic, phosphoric, fumaric, lactic, gluconic and tartaric acid or mixtures thereof.
Definition 15. Pharmaceutical composition according to any of the definitions 1 to 14, wherein when in the form of A or B, said composition additionally comprises one or more pharmaceutically acceptable excipients.
Definition 16. Pharmaceutical composition according to any of the definitions 1 to 5, wherein when in the form of C suitable for effervescent tablets or effervescent granules, said composition comprises one or more effervescent agents, preferably ascorbic acid and/or anhydrous citric acid and/or anhydrous sodium bicarbonate and/or sodium bicarbonate and/or sodium tartrate and/or sodium bitartrate.
Definition 17. Pharmaceutical composition according to definition 16, wherein when in the form of C, said composition additionally comprises one or more excipients, preferably one or more diluents and/or one or more lubricants and/or one or more binders and/or one or more sweeteners and/or one or more flavor enhancers.
Definition 18. Pharmaceutical composition according to any of the definitions 1 to 17, which additionally comprises one or more active substances other than ibuprofen or paracetamol or a pharmaceutically acceptable derivative thereof, for example other non-steroidal anti-inflammatory drugs for example acetylsalicylic acid and/or opioids for example codeine and/or anticonvulsants for example hyoscine and/or antihistamines for example chlorphenamine, orphenadrine and/or mucolytics for example acetylcysteine and/or sympathomimetics for example pseudoephedrine and/or vitamin C and/or caffeine.

It was surprisingly found that the composition of the aqueous solution of ibuprofen in the form of either sodium salt or lysinate and paracetamol comprising one or more solubilizing agents, preferably one or more cyclodextrins, was stable even at high temperatures for a long time.

It was surprisingly found that the composition of an aqueous solution of ibuprofen in the form of either sodium salt or lysinate and paracetamol comprising one or more solubilizing agents, preferably one or more cyclodextrins, exhibited even more increased solubilization of ibuprofen.

This new proposed combination of ibuprofen in the form of either sodium salt or lysinate and paracetamol and preferably cyclodextrin exhibits all the advantages of the prior art and indeed with unexpectedly improved stability and in particular improved solubility.

It was surprisingly found that the composition of an aqueous solution of ibuprofen comprising one or more solubilizing agents, especially one or more basic amino acids and optionally one or more cyclodextrins, was stable even at high temperatures for a long time.

It was surprisingly found that the composition of an aqueous solution of ibuprofen comprising one or more solubilizing agents, especially one or more basic amino acids and optionally one or more cyclodextrins, showed even more increased solubilization of ibuprofen.

This new proposed combination of ibuprofen with histidine and optionally with cyclodextrin of the present invention exhibits all the advantages of the prior art with unexpectedly improved stability and especially improved solubility.

In addition to the strong improvement in the solubility and non-immediate precipitation of the active substances of the composition, in the form of orally administrated forms, it was surprisingly found that the absorption of said active substances is improved on the one hand, and on the other hand the onset of their action is accelerated.

It was also surprisingly found that the ibuprofen, paracetamol and additionally cyclodextrin compositions of the present invention in the form of orally administrated forms, showed an unexpected improvement in the unpleasant taste, resulting not only from ibuprofen but also from the combination of ibuprofen and paracetamol.

This new proposed combination of ibuprofen-paracetamol with cyclodextrin of the present invention solves all the above problems of the state of the art, and in fact with unexpected, surprising results in term of improved solubility.

Furthermore, it was surprisingly found that the more is restricted the scope of the claims, the more intense are the effects and the advantages of the present invention.

In particular, the more intense are the effects and the advantages of the present invention were observed when ibuprofen was combined with paracetamol.

By the expression equivalent free ibuprofen we mean the free form of ibuprofen, i.e. not the salt form e.g. sodium or other complex e.g. with lysine.

The stable aqueous solution composition for use in intravenous infusion according to the present invention may further comprise isotonic agents, preferably sodium chloride.

The stable aqueous solution formulation for use in intravenous infusion according to the present invention can be sterilized by heat or by filtration.

The aqueous medium of the stable aqueous solution composition for use in intravenous infusion according to the present invention may be deoxygenated by a water-insoluble inert gas (N2).

The compositions of the present invention will be administered intravenously and are stable when stored for more than 24 months at room temperature, saving refrigeration costs during transport and storage and alleviating patient discomfort during administration. Furthermore, the compositions may even be stable when stored for more than 30 days at elevated temperatures, for example 70° C.

The compositions of the present invention may be prepared in solution and stored in transparent glass containers, for example glass ampoules, vials, cartridges, glass sealed vials or in stoppered glass vials or bottles of polymeric material such as polyethylene or in bags of soft polyethylene material, polyvinyl chloride or polypropylene. The glass should preferably be transparent to facilitate use.

The amounts of cyclodextrin may be of the same order of magnitude as those reported in CN101991540A, the entire contents of which are incorporated into the description of the present invention. For example, the cyclodextrin may be in an amount of from 100 to 600 mg per dose, e.g. 120, 200, 300, 400, 500 mg per dose.

The compositions of the present invention can be prepared by methods already known in the state of art.

The present invention is further described by the following indicative, but non-limiting examples.

Example 1: In a preferred embodiment the composition of the present invention comprises the following per 100 mL

| | | |
|---|---|---|
| Ibuprofen sodium | 331.97 | mg |
| Paracetamol | 1000 | mg |
| 2-hydroxypropyl-β-cyclodextrin | 666 | mg |
| EDTA | 10 | mg |
| Monothioglycerol | 10 | mg |
| Sodium chloride | 600 | mg |
| Sodium hydrogen phosphate | 35.6 | mg |
| Water suitable for injection | 100 | mL |

| | |
|---|---|
| Final pH (HCl or NaOH) 1M | 5.5-6.5 |

Suitable for intravenous injection

Example 2: In a preferred embodiment the composition of the present invention comprises the following per 100 mL

| | | |
|---|---|---|
| Ibuprofen lysinate | 512.7 | mg |
| Paracetamol | 1000 | mg |
| 2-hydroxypropyl-β-cyclodextrin | 666 | mg |
| EDTA | 10 | mg |
| Monothioglycerol | 10 | mg |
| Sodium chloride | 600 | mg |
| Sodium hydrogen phosphate | 35.6 | mg |
| Water suitable for injection | 100 | mL |

| | |
|---|---|
| Final pH (HCl or NaOH) 1M | 5.5-6.5 |

Suitable for intravenous injection

Example 3: In a preferred embodiment the composition of the present invention comprises the following per 100 ml

| | | |
|---|---|---|
| Ibuprofen sodium | 331.97 | mg |
| Paracetamol | 1000 | mg |
| 2-hydroxypropyl-β-cyclodextrin | 666 | mg |
| EDTA | 10 | mg |
| Monothioglycerol | 10 | mg |
| Thiamine hydrochloride | 10 | mg |
| Sodium chloride | 600 | mg |
| Sodium hydrogen phosphate | 35.6 | mg |
| Water suitable for injection | 100 | mL |

| | |
|---|---|
| Final pH (HCl or NaOH) 1M | 5.5-6.5 |

Suitable for intravenous injection

Example 4: In a preferred embodiment the composition of the present invention comprises the following per mL

| | |
|---|---|
| Ibuprofen | 100 mg/mL |
| Histidine | 74 mg/mL |
| Water suitable for injection | 1 mL |
| Sodium carbonate | Qs to pH = 7.4 ± 0.5 |

Suitable for intravenous injection

Example 5: In a preferred embodiment the composition of the present invention comprises the following per mL

| | |
|---|---|
| Ibuprofen | 100 mg/mL |
| Histidine | 74 mg/mL |
| 2-hydroxypropyl-β-cyclodextrin | 712 mg/mL |
| Water suitable for injection | 1 mL |
| Sodium carbonate | Qs to pH = 7.6 ± 0.5 |

Suitable for intravenous injection

Example 6: In a preferred embodiment the composition of the present invention comprises the following per ml

| | |
|---|---|
| Ibuprofen | 100 mg/mL |
| Hisitidine | 74 mg/mL |
| 2-hydroxypropyl-β-cyclodextrin | 712 mg/mL |
| monothioglycerol | 10 mg/mL |
| EDTA | 10 mg/mL |
| Water suitable for injection | 1 mL |
| Sodium carbonate | Qs to pH = 7.6 ± 0.5 |

Suitable for intravenous injection

Example 7: In a preferred embodiment the composition of the present invention comprises the following per ml

| | |
|---|---|
| Ibuprofen | 3 mg/mL |
| Histidine | 74 mg/mL |
| Paracetamol | 10 mg/mL |
| 2-hydroxypropyl-β-cyclodextrin | 712 mg/mL |
| Water suitable for injection | 1 mL |
| Sodium carbonate | Qs to pH = 7.6 ± 0.5 |

Suitable for intravenous injection with infusion

Example 8: In an embodiment the composition of the present invention comprises the following per effervescent tablet

| | |
|---|---|
| ibuprofen | 400 mg |
| paracetamol | 500 mg |
| 2-hydroxypropyl-β-cyclodextrin | 500 mg |
| arginine | 250 mg |
| sodium bicarbonate | 1000 mg |
| sodium bitartrate | 1200 mg |
| sodium saccharin | 40 mg |
| polyvidone | 90 mg |
| flavor (powder) | 200 mg |

The compositions of Example 1 are formulated into effervescent tablets.

Example 9: In an embodiment the composition of the present invention comprises the following per sachet

| | |
|---|---|
| ibuprofen | 200 mg |
| paracetamol | 500 mg |
| 2-hydroxypropyl-β-cyclodextrin | 300 mg |
| sodium bicarbonate | 1000 mg |
| sodium bitartrate | 1200 mg |
| Vitamin C | 500 mg |
| sodium saccharin | 40 mg |
| polyvidone | 90 mg |
| flavor (powder) | 200 mg |

The compositions of Example 1 are formulated into effervescent granules.

Example 10: In an embodiment the composition of the present invention comprises the following per dose of 5 ml

| | | |
|---|---|---|
| ibuprofen | 150 | mg |
| paracetamol | 100 | mg |
| 2-hydroxypropyl-β-cyclodextrin | 120 | mg |
| xanthan gum | 7.5 | mg |
| glycerol | 500 | mg |
| sodium benzoate | 12.5 | mg |
| citric acid | 10 | mg |
| sodium saccharin | 2.5 | mg |
| polysorbate 80 | 15 | mg |
| microcrystalline cellulose | 60 | mg |
| water | qs to 5 | mL |
| strawberry flavor | 12.5 | mg |
| colouring agent | 0.5 | mg |
| sorbitol solution 70% | 2000 | mg |
| Caramellose sodium | 7.5 | mg |
| EDTA | 2.5 | mg |

The compositions of Example 1 are formulated into syrup

The compositions of the present examples 1-10 exhibit all the advantages of the present invention mentioned above, e.g. solubility, improved taste (in the cases of the orally administrated examples 8-10), improved absorption and faster onset of action.

In addition, we prepared the compositions of the above examples 1-3 and 5-10 in the absence of cyclodextrin and found clearly lower solubility with almost immediate precipitation of the active substances.

1. A pharmaceutical composition comprising ibuprofen or a pharmaceutically acceptable derivative thereof, and one or more solubilizing agents, wherein the pharmaceutical composition is in the form of:
A) an aqueous solution for use in intravenous administration comprising the ibuprofen in the form of sodium salt or lysinate salt, and further comprising paracetamol,
B) an aqueous solution for use in intravenous administration, wherein the one or more solubilizing agents are one or more essential amino acids and, optionally, one or more cyclodextrins, or
C) a composition for use in oral administration, further comprising paracetamol and cyclodextrin, wherein the composition is in the form of;
i) effervescent tablets or effervescent granules,
ii) an oral suspension or syrup or oral solution, or
iii) soft capsules.

2. The pharmaceutical composition of claim 1, wherein the ibuprofen is in the form of the sodium salt.

3. The pharmaceutical composition of claim 1, wherein the amount of the ibuprofen ranges from 1 to 1000 mg, preferably from 150 the 600 mg or 200 the 500 mg or 300 to 400 mg.

4. The pharmaceutical composition of claim 1,
wherein the pharmaceutical composition is in the form of the aqueous solution A or the aqueous solution B, and
wherein the concentration of equivalent free ibuprofen ranges from 1 to 400 mg/mL.

5. The pharmaceutical composition of claim 1 further comprising paracetamol.

6. The pharmaceutical composition of claim 1,
wherein the pharmaceutical composition is in the form of the aqueous solution A, and
wherein the concentration of the paracetamol ranges from 0.20% to 10% m/v.

7. The pharmaceutical composition of claim 1,
wherein the pharmaceutical composition is in the form of the aqueous solution A or the aqueous solution B,
wherein the pharmaceutical composition comprises one or more cyclodextrins, and
wherein the concentration of the one or more cyclodextrins ranges from 0.20% m/v to 19% m/v.

8. The pharmaceutical composition of claim 1,
wherein the pharmaceutical composition is in the form of the aqueous solution A or the aqueous solution B, wherein the pharmaceutical composition comprises one or more cyclodextrins, and
wherein the concentration of the one or more cyclodextrins ranges from 10 to 2000 mg/mL.

9. The pharmaceutical composition of claim 1,
wherein the pharmaceutical composition comprises one or more essential amino acids,
wherein the pharmaceutical composition is in the form of the aqueous solution A or the aqueous solution B, and
wherein the concentration of the one or more essential amino acids ranges from 1 to 400 mg/L.

10. The pharmaceutical composition of claim 1,
wherein the pharmaceutical composition is in the form of the aqueous solution A or the aqueous solution B,
wherein the pharmaceutical composition comprises one or more essential amino acids and one or more cyclodextrins,
wherein the concentration of the one or more essential amino acids ranges from 0.5 to 200 mg/L, and
wherein the concentration of the one or more cyclodextrins ranges from 10 to 2000 mg/mL.

11. The pharmaceutical composition of claim 1,
wherein the pharmaceutical composition is in the form of the aqueous solution A or the aqueous solution B,
wherein the pharmaceutical composition further comprises a derivative bearing at least one thiol functional group selected from the group consisting of thioglycerols, cysteine, acetylcysteine, thioglycolic acid and/or salts thereof, dithiothreitol, reduced glutathione, thiolactic acid and/or salts thereof, and mercaptoethanesulfonic acid, and
wherein the concentration of the derivative bearing the at least one thiol functional group ranges from 0.001% to 0.5% m/v, from 1.5% to 3% m/v, or from 2% to 2.5% m/v.

12. The pharmaceutical composition of claim 1,
wherein the pharmaceutical composition is in the form of the aqueous solution A or the aqueous solution B,
wherein the pharmaceutical composition further comprises a chelating agent selected from the group consisting of EDTA, nitrilotriacetic acid, ethylenediamine-N,N′-dipropionic acid, ethylenediamine-tetra-(methylene phosphoric acid)), 2,2′ (ethylenediamino)-dibutyl acid, bis(2-aminoethyl ether)-N,N,N′ethylene glycol, N′-tetraacetic acid, and salts thereof,
and wherein the concentration of the chelating agent ranges from 0.001% to 0.5% m/v, from 1.5% to 3% m/v, or from 2% to 2.5% m/v.

13. The pharmaceutical composition of claim 1,
wherein the pharmaceutical composition is in the form of the aqueous solution A or the aqueous solution B,
wherein the pharmaceutical composition further comprises one or more derivatives selected from thiamine salts, and
wherein the concentration of the thiamine salts ranges from 0.001% m/v to 0.2% m/v.

14. The pharmaceutical composition of claim 1,
wherein the pharmaceutical composition is in the form of the aqueous solution A or the aqueous solution B,
wherein pH of the pharmaceutical composition is between 4 and 8,
wherein the pH is adjusted by using a suitable alkalizing agent and/or by at least one acid and its ionized form, and
wherein the at least one acid is selected from the group consisting of citric, malic, acetate, sorbic, phosphoric, fumaric, lactic, gluconic, tartaric acid, and mixtures thereof.

15. The pharmaceutical composition of claim 1,
wherein the pharmaceutical composition is in the form of the aqueous solution A or the aqueous solution B, and
wherein the pharmaceutical composition further comprises one or more pharmaceutically acceptable excipients.

16. The pharmaceutical composition of claim 1,
wherein the pharmaceutical composition is in the form of the composition C,
wherein the composition C is in the form of the effervescent tablets or the effervescent granules, and
wherein the effervescent tablets or the effervescent granules comprise one or more effervescent agents.

17. The pharmaceutical composition of claim 16, wherein the composition C further comprises one or more excipients.

18. The pharmaceutical composition of claim 1 further comprising one or more active substances other than ibuprofen, paracetamol, or a pharmaceutically acceptable derivative thereof.

19. The pharmaceutical composition of claim 18, wherein the one or more active substances are selected from the group consisting of acetylsalicylic acid, opioids, codeine, anticonvulsants, hyoscine, antihistamines, chlorphenamine, orphenadrine, mucolytics, acetylcysteine, sympathomimetics, pseudoephedrine, vitamin C, and caffeine.

20. The pharmaceutical composition of claim 5, wherein the ratio of ibuprofen:paracetamol is 600 mg:1000 mg, 500 mg:1000 mg, 500 mg:500 mg, 400 mg:1000 mg, 400 mg:500 mg, 300 mg:1000 mg, 300 mg:500 mg, 200 mg:1000 mg, 200 mg:500 mg, 200 mg:200 mg, 200 mg:100 mg, 150 mg:1000 mg, 150 mg:500 mg, 150 mg:200 mg, or 150 mg:100 mg.
